(19) United States
(12) Patent Application Publication
(10) Pub. No.: US 2020/0261025 A1
(43) Pub. Date: Aug. 20, 2020

(54) SYSTEM AND METHOD FOR MAKING AND IMPLANTING HIGH-DENSITY ELECTRODE ARRAYS

(71) Applicant: Board of Regents, The University of Texas System, Austin, TX (US)

(72) Inventors: Chong XIE, Austin, TX (US); Lan LUAN, Austin, TX (US); Zhengtuo ZHAO, Austin, TX (US); Xiaoling WEI, Austin, TX (US)

(21) Appl. No.: 16/645,205

(22) PCT Filed: Sep. 7, 2018
(86) PCT No.: PCT/US18/49853

§ 371 Date: Mar. 6, 2020

Related U.S. Application Data
(60) Provisional application No. 62/555,798, filed on Sep. 8, 2017.

(51) Int. Cl. A61B 5/00; A61B 5/0478; G06F 3/01

(52) U.S. Cl. CPC A61B 5/6868; A61B 5/0478; G06F 3/015; A61B 2562/164; A61B 2562/0285; A61B 2562/043; A61B 2560/0487

(57) ABSTRACT

A flexible probe includes a probe body having a distal end and a proximal end, and comprises at least one flexible nanoelectronic thread having a distal end and a proximal end and a length therebetween, the nanoelectronic thread comprising at least one electrode positioned on a top surface of the nanoelectronic thread, and at least one nanoscale wire is partially encapsulated, having a first portion electrically connected to at least one of the electrodes and a second portion surrounded by a dielectric insulator, the probe further including at least one external trace electrically connected to the at least one nanoscale wire at the proximal end of the at least one nanoelectronic thread, the at least one external trace having a width larger than the width of the at least one nanoscale wire, and an interface connector near the proximal end of the probe body, electrically connected to the at least one electrode via the at least one nanoscale wire and the at least one external trace, wherein the at least one nanoscale wire has a width less than 250 nm, and wherein the nanoelectronic thread has a cross-sectional area that is less than 30 μm². Methods of fabricating and implanting a flexible probe are also described.

### CROSS-REFERENCE TO RELATED APPLICATIONS

This application is a '371 national phase entry of PCT application no. PCT/US18/49853, filed on Sep. 7, 2018, which claims priority to U.S. provisional patent application No. 62/555,798, filed on Sep. 8, 2017, both of which are incorporated herein by reference in their entireties.

### STATEMENT REGARDING FEDERALLY SPONSORED RESEARCH OR DEVELOPMENT

This invention was made with government support under Grant no. W81XWH-16-1-0580 awarded by the Army Medical Research and Material Command, ARMY/MRMC. The government has certain rights in the invention.

### BACKGROUND OF THE INVENTION

Implanted neural probes such as microwires, tetrodes, and silicon-based microelectrodes are among the most important techniques in both fundamental and clinical neuroscience. Scientifically, they remain the only option to temporally resolve the fastest electrophysiological activities of individual neurons, thus providing critical information to dissect neural circuitry. Clinically, neural electrodes have been successfully used in the treatment of neurological disorders such as Parkinson's disease, epilepsy, and obsessive compulsive disorder. Moreover, neural electrodes allow for direct communication between brain and man-made devices, which can enable applications such as human brain-machine interfaces and neuroprosthetics.

However, these conventional neural electrical probes typically have dimensions substantially larger than neurons and capillaries, which fundamentally precludes the possibility of interrogating the whole neuronal population in a functional brain region. For example, microwire electrodes, tetrodes, and Utah arrays host only one recording site at the tip of each wire, and therefore cannot simultaneously record neural activity at multiple depths. Micro-Electro-Mechanical System (MEMS)-based silicon probes have significantly increased the number of recording sites on a single probe. However, these silicon probes typically have cross-sectional areas around or greater than 10 μm², which yields a volume per electrode similar to that of microwires and tetrodes, at least two orders of magnitude larger than the average size of a neuronal soma. In addition, these probes are strongly invasive to living brain tissue. Therefore, their highest implantation density is typically limited to maintain tissue vitality, by allowing at most 1-2% of the enclosed volume to be occupied by the electrode array. Therefore, the smallest inter-probe spacing is limited to at least several hundred microns for both microwire array and silicon probes.

In an effort to further reduce electrode size and increase packing density, advanced lithography techniques such as electron-beam lithography (EBL) have been used to fabricate silicon-based microelectrodes that enable closely-packed recording sites along the length of a probe. However, these nanofabricated probes have similar dimensional limits on implantation density. Ultra-flexible nanoelectronic neural probes, using a substrate-less, multi-layer layout, markedly reduce the cross-section to the subcellular range, but demonstrate limited electrode density along the probe due to the fabrication resolution of planar photolithography techniques.

Understanding brain functions at the circuit level requires time-resolved simultaneous measurement of a large number of densely distributed neurons, which remains a great challenge for current neural technologies. Currently available probes preclude the high implant density that is necessary for mapping large neuronal populations with full coverage.

Thus, there is a need in the art for an improved intracortical electrode array. The present invention satisfies that need.

### SUMMARY OF THE INVENTION

In one aspect, a probe comprises a probe body having a distal end and a proximal end, at least one flexible nanoelectronic thread extending from the distal end of the probe body, the nanoelectronic thread comprising at least one electrode positioned on a top surface of the nanoelectronic thread, and at least one nanoscale wire having a first portion electrically connected to the at least one electrode and a second portion encapsulated by a dielectric insulator, at least one external trace electrically connected to the at least one nanoscale wire at a proximal end of the at least one nanoelectronic thread, and an interface connector near the proximal end of the probe body, electrically connected to the at least one electrode via the at least one nanoscale wire and the at least one external trace, wherein the at least one nanoscale wire has a width less than 1 μm, and wherein the nanoelectronic thread has a cross-sectional area that is less than 100 μm². In one embodiment, the at least one electrode comprises a plurality of electrodes positioned on the top surface of the flexible nanoelectronic thread, the plurality of electrodes being evenly spaced along a length of the nanoelectronic thread, and the at least one nanoscale wire comprises a plurality of nanoscale wires, the nanoscale wires having a pitch of less than 500 nm. In one embodiment, the plurality of electrodes are arranged in a linear array, and wherein the cross-sectional area of the flexible nanoelectronic thread is less than 10 μm².

In one embodiment, the at least one electrode comprises at least one array of electrodes, the electrodes in the array being separated by a distance of less than 10 μm. In one embodiment, each of the electrodes has a sensing surface having an area less than 100 μm². In one embodiment, the at least one nanoelectronic thread comprises at least 8 nanoscale wires. In one embodiment, the at least one nanoelectronic thread comprises at least 16 nanoscale wires. In one embodiment, the at least one nanoelectronic thread further comprises a hole having a diameter of less than 10 μm. In one embodiment, the at least one external trace has a width greater than the width of the at least one nanoscale wire.

In another aspect, a method of fabricating a flexible probe comprises the steps of patterning at least one external trace on a substrate using photolithography, fabricating a flexible nanoelectronic thread using a process comprising the steps of depositing a sacrificial layer on a substrate, depositing a first insulating layer on top of the sacrificial layer, patterning at least one nanoscale wire on top of the insulating layer using electron beam lithography, depositing a second insulating layer on top of the at least one nanoscale wire, thereby encapsulating at least a first portion of the at least one nanoscale wire and leaving at least a second portion of the at least one nanoscale wire exposed, patterning at least one electrode on top of the second insulating layer, wherein the at least one electrode is in electrical contact with the second portion of the at least one nanoscale wire, and removing the sacrificial layer and releasing the finished probe from the substrate, wherein the electron beam lithography and photolithography are overlapped by at least 4 μm in all directions in all layers.

In one embodiment, the sacrificial layer comprises nickel and is removed with a nickel etchant. In one embodiment, the first and second insulating layers comprise SU8. In one embodiment, the electron beam lithography is controlled at a resolution of 0.5 μC/cm².

In another aspect, a method of implanting a flexible probe in a tissue of a subject comprises the steps of attaching a flexible probe to a rigid implantation assist device at an attachment point, inserting the flexible probe into a tissue of a subject by piercing the tissue with the rigid implantation device and inserting the rigid implantation assist device such that the attachment point is within the tissue, detaching the flexible probe from the rigid implantation assist device, and withdrawing the rigid implantation device from the tissue.

In one embodiment, the step of attaching further comprises the step of inserting a micro post on the rigid implantation device into a hole near a distal end of the flexible probe. In one embodiment, the rigid implantation device and the flexible probe are inserted at a speed of 5 μm/second. In one embodiment, the flexible probe is attached to the rigid implantation device via a chemical bond. In one embodiment, the chemical bond comprises polyethylene glycol. In one embodiment, the detaching step further comprises dissolving the polyethylene glycol. In one embodiment, the rigid implantation assist device comprises a linear actuator and a carrier block slidably attached to a linear guide and fixedly attached to the flexible probe, and wherein the method further comprises actuating the linear actuator thereby implanting the flexible probe into the tissue. In one embodiment, the linear actuator comprises a solenoid. In another aspect, a probe is made according to one or more methods described above.

In another aspect, a system for implanting a flexible probe in a tissue of a subject comprises a flexible probe and a rigid implantation assist device, wherein the flexible probe is configured to engage the rigid implantation device. In one embodiment, the rigid implantation assist device comprises a post configured to be inserted into a hole positioned at the distal end of the flexible probe. In one embodiment, the rigid implantation device comprises a linear actuator and a carrier block slidably attached to a linear guide and fixedly attached to the flexible probe.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a photograph of one embodiment of an intracortical electrode array;

FIG. 1B is a cross-sectional diagram of an embodiment of an intracortical electrode array;

FIG. 2 is a photograph of another embodiment of an intracortical electrode array;

FIG. 3 is a photograph of another embodiment of an intracortical electrode array;

FIG. 4 is a cross-sectional diagram of an embodiment of an intracortical electrode array;

FIG. 5 is a scanning-electron microscope image and corresponding height graph of a pair of electrodes in an intracortical electrode array;

FIG. 6, comprising FIG. 6A through FIG. 6C, is a set of photographs of another embodiment of an intracortical probe;

FIG. 7 is a flow diagram of a method of fabricating an intracortical electrode array;

FIG. 8A is a set of photographs of an intracortical probe;

FIG. 8B is a set of photographs of various embodiments of intracortical electrode arrays;

FIG. 8C is a graph of implantation footprints and sizes of various intracortical probes;

FIG. 9A is a diagram of a system and method for implantation of an intracortical electrode array;

FIG. 9B is a set of photographs of the tips of exemplary intracortical electrode arrays, highlighting the implantation assistance features;

FIG. 9C is a diagram of a system and method for implantation of an intracortical electrode array;

FIG. 10 is a set of diagrams of various parallel implantation methods;

FIG. 11, comprising FIG. 11A through FIG. 11G, is a set of graphs of signals obtained through experimentation with intracortical electrode arrays;

FIG. 12, comprising FIG. 12A through FIG. 12H, is a set of images and photographs of various parallel arrangements of intracortical probes;

FIG. 13, comprising FIG. 13A through FIG. 13F is a set of images and photographs of various parallel arrangements of intracortical probes;

FIG. 14, comprising FIG. 14A through FIG. 14H, is a set of photographs of various parallel arrangements of microconduits to guide the implantation of intracortical probes;

FIG. 15, comprising FIG. 15A through FIG. 15D, is a set of graphs and photographs related to an implantation experiment for an intracortical electrode array; and

FIG. 16, comprising FIG. 16A through FIG. 16D, is a set of photographs of brain tissue after prolonged exposure to some intracortical probes of the present invention.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

Embodiments of the present invention relate to an ultra-flexible electrode array platform that can reliably detect and stimulate the electrical activity of individual or groups of cells (e.g., neurons, peripheral nerves, etc.). In certain instances, the platform allows for detection and/or stimulation with minimal immune responses over chronic time scales of months to years. Further, the present invention provides a strategy to chronically implant these electrodes in living tissue at high-packing density in three dimensions. The present invention is based, in part, upon the demonstrated ability to drastically reduce the dimensions of the probe by multiple orders of magnitude from currently-used microelectrodes, and the ability to integrate these electrodes with a minimally-invasive implantation mechanism for high-density, large-scale, parallel delivery into living tissue. The separation of electrodes in all three dimensions can be reduced to sub-100 μm, which will allow for complete coverage of all enclosed cells in a given volume. Because the ultra-flexible electrodes also have great long-term biocompatibility, the platform of the present invention also enables stable long-term recording and stimulation without the artifact of scar-encapsulation, continuous blood leakage and other tissue response to conventional electrodes. Therefore, this platform will open new opportunities for neuroscience research, clinical therapeutic applications for neuronal diseases, and development of brain machine interfaces. Probes of the present invention may be used for example to detect and monitor signals in a subject having a disability or condition, including but not limited to paralysis, amputation, blindness, or hearing loss. Other relevant conditions include neurological disorders, including but not limited to epilepsy, depression, addiction disorder, or neurodegenerative disorders such as Alzheimer's disease, Parkinson's disease, Huntington's disease, amyotrophic lateral sclerosis (ALS), and cerebrovascular diseases such as stroke. Probes of the present invention may also be used for fundamental and applied neuroscience research in experimental animal models in laboratories.

The present invention generally relates to nanoscale wires, nanoelectronic threads comprising multiple nanoscale wires and multiple electrodes, and, to probes comprising one or more nanoelectronic threads for use in detecting and/or stimulating electrical activity of biological tissue, including but not limited to the brain, spinal cord, heart, or peripheral nerves. Probes according to the present invention may be formed from relatively flexible materials, and in some cases, the probes may comprise nanoscale wires or other electronic components. Other embodiments are generally directed to systems and methods of making, using, or implanting such probes, kits involving such probes, and the like.

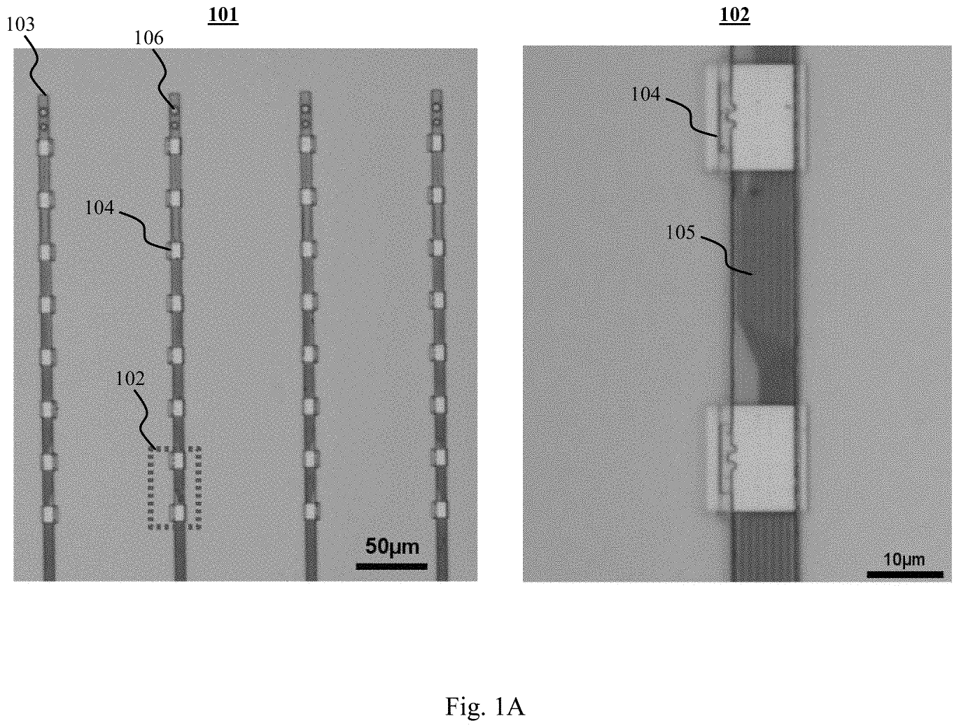

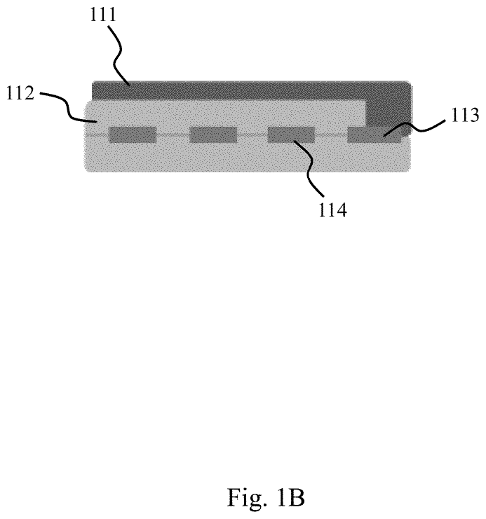

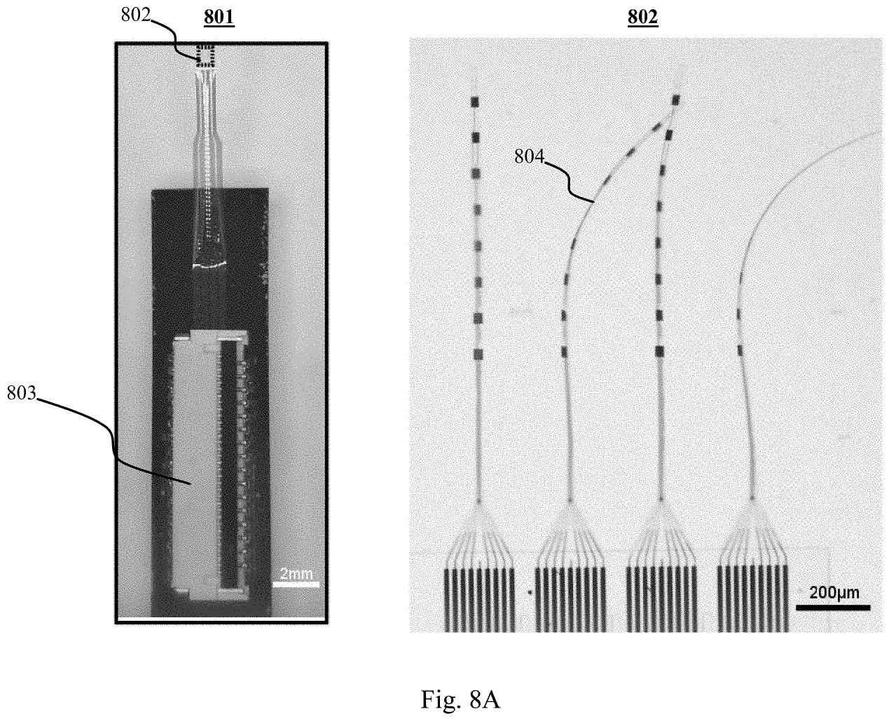

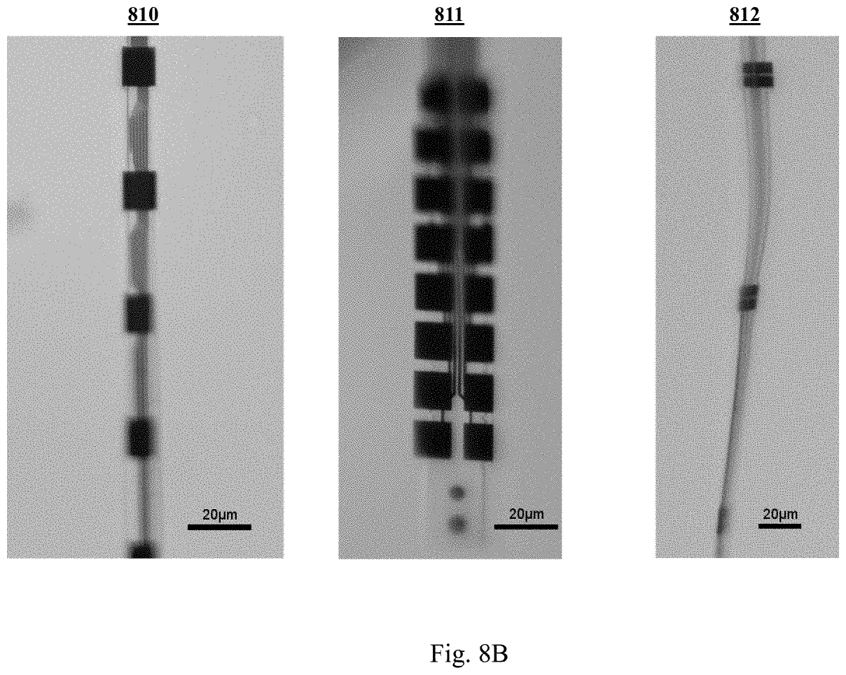

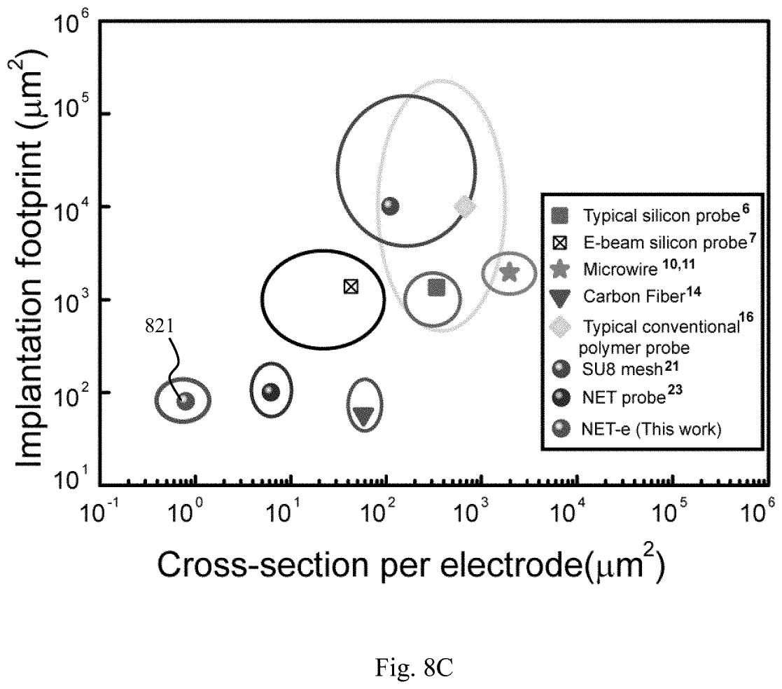

The various components described above may best be understood with reference to the figures. With reference to FIG. 1A, element 101 depicts four nanoelectronic threads 103, each comprising eight electrodes 104. In section view 102, individual nanoscale wires 105 are visible. FIG. 8A depicts a higher-level view of a probe in element 801. As shown in section view 802, a probe comprises one or more nanoelectronic threads 804, each of which comprises one or more electrodes and one or more nanoscale wires.

One aspect of the present invention is generally directed to a probe for insertion into a tissue, or other material. The probe can be fully or partially inserted into the tissue or other material. The probe may be used to determine a property of the tissue or other material, and/or provide an electrical signal to the tissue or other material. This may be achieved using one or more nanoscale wires on the probe. For example, in certain embodiments, a probe comprising one or more electrodes may be inserted into an electrically-active tissue, such as the heart or the brain, and the one or more electrodes may be used to determine electrical properties of the tissue, e.g., action potentials or other electrical activity. In some cases, the one or more nanoelectronic threads of the probe are relatively porous to allow cells, axons, blood vessels, or other biological structures to infiltrate the threads. This may be useful for long-term applications, for example, where the probe is to be inserted and used for days, weeks, months, or years within the tissue. In some embodiments, neurons or cardiac cells may be able to grow around and/or into the one or more threads while they are inserted into the brain or the heart over extended periods of time.

In one embodiment, a system of the present invention comprises a probe comprising one or more nanoelectronic threads. In certain instances, elements of the probe are manufactured with a substrate-less device architecture and using electron-beam lithography to define the smaller features. Nanoelectronic thread of the present invention may comprise a densely-packed electrode array and versatile design patterns, with each thread comprising one or more electrodes. In some embodiments, each thread comprises at least eight electrodes. Each probe may comprise one or more nanoelectronic threads, wherein some embodiments of individual threads have a cross-sectional area of less than about 10 μm². In other embodiments, individual threads have a cross-sectional area of less than about 5 μm², less than about 2 μm², or less than about 1 μm².

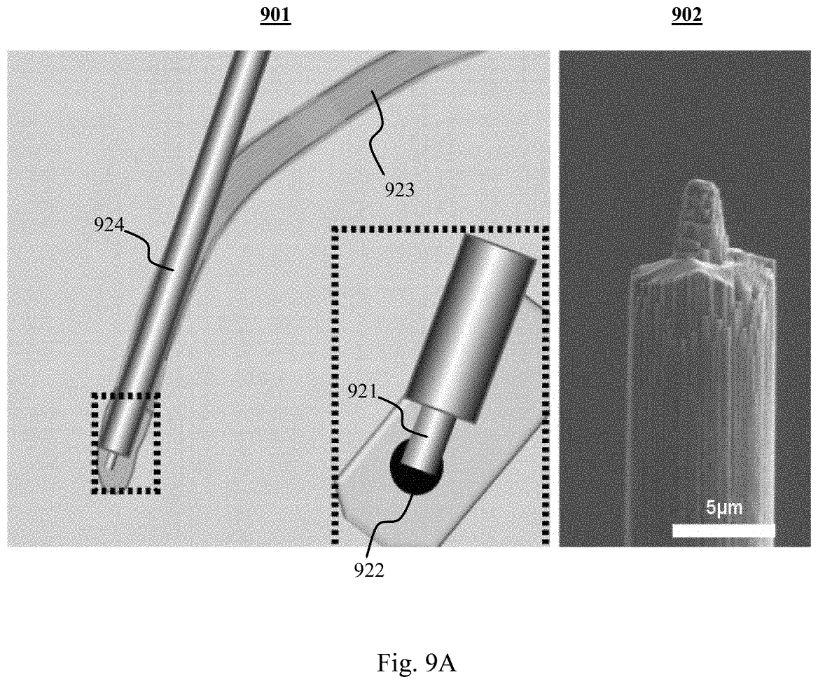

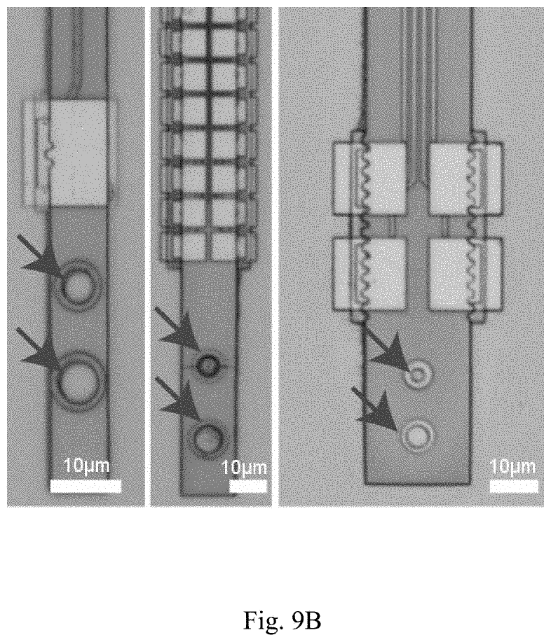

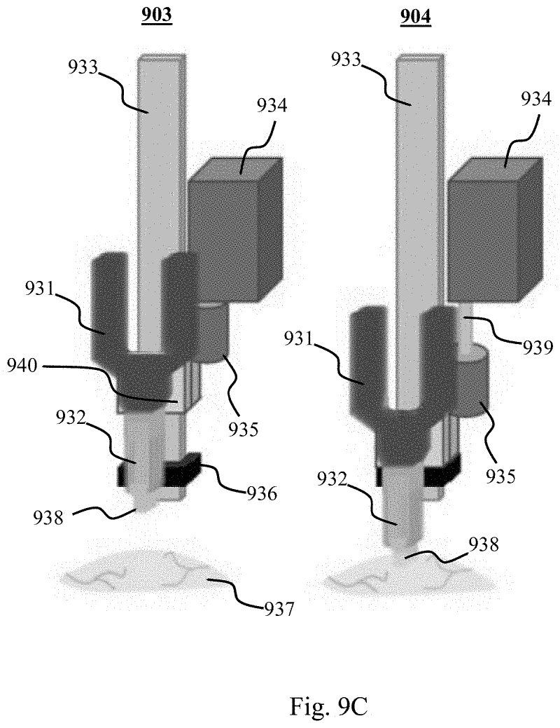

Referring now to FIG. 1-FIG. 3, images of various embodiments of the nanoelectronic thread of the present invention are shown. FIG. 1A comprises system view 101 and detail view 102, which is shown as an inset of system view 101. The embodiment of FIG. 1A shows multiple threads 103 each having a linear array of electrodes 104. In detail view 102, the nanoscale wires 105 can be seen contained within an individual thread. Electrodes 104 are exposed, to make physical and electrical contact with cells in tissue of a subject. In one embodiment, each electrode is positioned to make electrical contact with an individual neuron in the brain of a subject. In other embodiments, each electrode is positioned to make contact with a neuron in the central nervous system, for example in the brain or spinal cord, or in the peripheral nervous system, including but not limited to the vagus nerve, the sciatic nerve, the median nerve, or the optic nerve. In certain embodiments, each electrode is positioned to make contact with a cell in other body tissue, including but not limited to cardiac tissue, muscle tissue, and the like. Nanoscale wires 105 are insulated, for example by a mask layer, to allow conduction of signals from various electrodes 104 without nanoscale wires 105 separately making electrical contact with cells. In some embodiments, nanoelectronic threads of the present invention comprise one or more holes 106 at the distal ends of the thread. Holes 106 may engage with an implantation assist device, for example as shown and described below in FIG. 9A. The cross-sectional area of each thread shown in FIG. 1A is approximately 8 μm×0.8 μm. Various embodiments of nanoelectronic threads of the present invention may have cross-sectional areas of less than about 100 μm², less than about 50 μm², less than about 30 μm², or less than about 10 μm².

FIG. 1B depicts an approximation of the cross section of the linear array shown in FIG. 1A. Shown in FIG. 1B is an electrode 111, positioned on top of dielectric insulator 112, in which multiple nanoscale wires 113, 114 are shown. As shown in FIG. 1B, nanoscale wire 113 is in electrical contact with electrode 111, and thus carries electrical signals to and from electrode 111 as needed. Nanoscale wire 114 is not in contact with electrode 111, therefore nanoscale wire 114 is in electrical contact with another electrode (not shown) in the linear array.

While in vivo components of probes of the present invention are ideally minimized, ex vivo components may be larger as convenient to promote reliability and ease of use. In one embodiment, the ex vivo component of a probe of the present invention is about 5 mm×30 mm, but in other embodiments it might be 10 mm×30 mm, 30 mm×30 mm, or 2 mm×10 mm, or any other suitable size. By contrast, the in vivo components, i.e. the one or more nanoelectronic threads, ideally have minimal cross-sectional area, but may be long if the tissue sought to be instrumented is deep within the subject. In one embodiment, the nanoelectronic threads are differently sized in order to maximize geometric granularity. In various embodiments, the one or more nanoelectronic threads have a length in the range of about 0.1 mm to about 10 cm. For example, in certain embodiments, the one or more nanoelectronic threads are 0.5 mm long, 1 mm long, 2 mm long, 5 mm long, or multiple centimeters long. The individual nanoelectronic threads on a given probe need not be the same length as one another, and each thread may be a different length from any other thread on the same probe as needed.

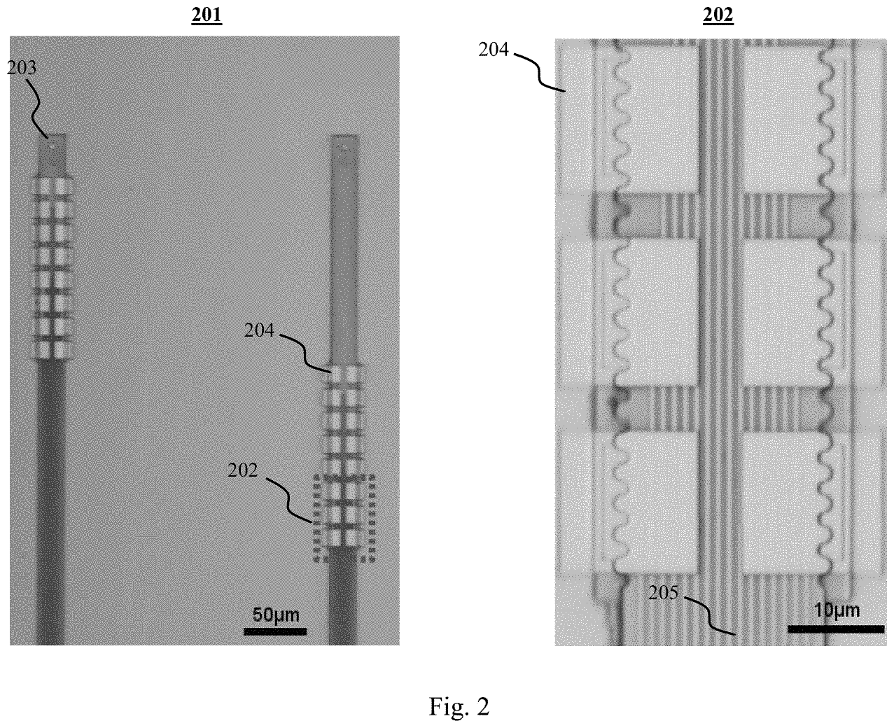

Referring now to FIG. 2, an oversampling array of electrodes on a nanoelectronic thread is shown. Similarly to FIG. 1A, FIG. 2 comprises a system-level view 201 depicting two strands 203 of nanoelectronic thread, each having a continuum of sixteen individually-addressed electrodes 204. As shown in detail view 202, each strand 203 further comprises multiple nanoscale wires 205.

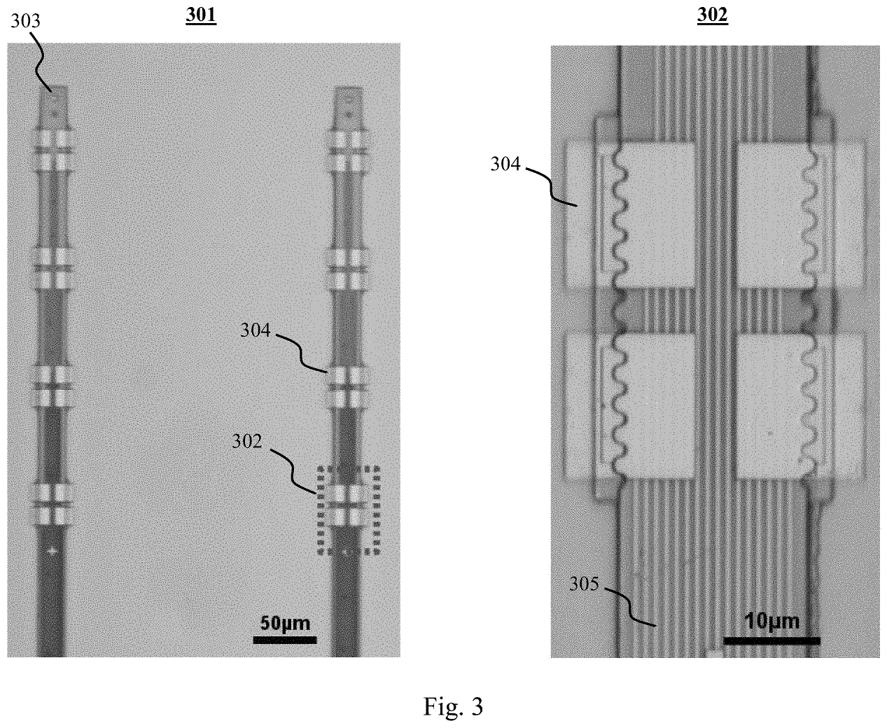

Referring now to FIG. 3, a further embodiment of the nanoelectronic thread of the present invention is shown, comprising system-level view 301 and detail view 302. In this embodiment, each thread 303 comprises multiple arrays of electrodes (each array sometimes referred to as a tetrode), each array comprising four electrodes 304. In some embodiments, the tetrodes are spaced 100 μm apart along the thread. Detail view 302 depicts a single array of four electrodes 304, as well as visible nanoscale wires 305. The embodiments of FIGS. 2 and 3 have electrode spacing smaller than their detection range to enable spatial oversampling in action potential recording. In various embodiments of probes of the invention, individual electrodes in an array may be spaced less than about 20 μm apart, less than about 10 μm apart, or less than about 5 μm apart. To minimize cross-sectional area, a multi-layer architecture is used with no substrate, where nanoscale wires and electrodes are fabricated on different layers and are separated by insulating layers. Each nanoscale wire may have a width of less than about 1 μm, less than about 500 nm, or less than about 250 nm.

The number and spacing of electrodes per thread and number of threads per probe may vary depending on the specific application. Each thread may have one electrode, or may have as many as 20, 50, 100, 300, or 500 electrodes. The maximum number of electrodes per thread is typically governed by the number of nanoscale wires necessary to connect each electrode to the probe. More wires means a larger thread cross-sectional area, which, in certain instances, increases the likelihood of negative impacts on the subject tissue due to implantation. In the embodiments shown in the figures, one layer of nanoscale wires is used, but multiple layers of nanoscale wires could be used in order to minimize the cross-sectional area per electrode. In one embodiment, the cross sectional area tapers down from the proximal end of the thread to the distal end of the thread, but in other embodiments the cross sectional area is approximately the same along the length of the thread. Each probe may also comprise a single thread or multiple threads. The number of threads per probe is limited only by the implantation footprint, which varies widely among subjects and implantation locations. Multi-thread embodiments described in the present disclosure are therefore not intended to limit the number of threads per probe in any way. Where multiple threads are used, the threads may be spaced apart from each other at a distance of about 10 μm apart to about 1 mm apart. For example, in certain instances, the threads may be about 15 μm apart, about 20 μm apart, about 25 μm apart, about 30 μm apart, about 35 μm apart, about 40 μm apart, about 45 μm apart, about 50 μm apart, about 75 μm apart, about 100 μm apart, about 150 μm apart, about 200 μm apart, about 250 μm apart, about 300 μm apart, about 400 μm apart, about 500 μm apart, about 750 μm apart, or about 1 mm apart. Threads may be spaced evenly or irregularly as desired for electrode positioning.

Individual electrodes may be evenly spaced apart as shown in FIG. 1A, or may be grouped together in closely-packed arrays as in FIGS. 2 and 3. The spacing between individual electrodes and arrays may be constant or variable as needed. In certain embodiments, the electrodes may be spaced apart from each other at a distance of about 50 μm to about 1 mm apart. For example, where electrodes are not grouped together, they may be spaced for example 100 μm apart, 200 μm apart, 500 μm apart, or any reasonable spacing within the constraints dictated by the subject tissue being measured.

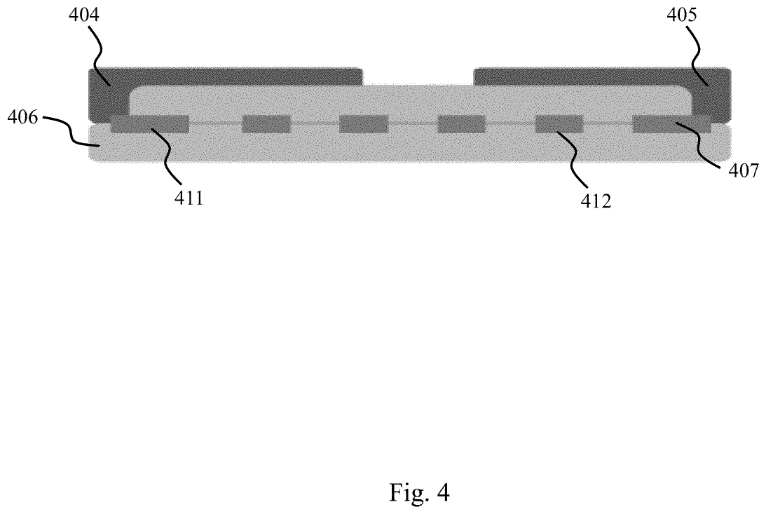

FIG. 4 shows an approximation of the cross section of a strand of nanoelectronic thread comprising an array of electrodes, such as the embodiment of FIG. 2 or 3. Shown in FIG. 4 are two electrodes 404 and 405, a dielectric insulating substrate 406, and a plurality of nanoscale wires 407, 411, and 412. Nanoscale wires 407 and 411 are in electrical contact with electrodes 405 and 404, respectively. Nanoscale wire 412 is not connected to either electrode 404 or 405, and is in electrical contact with another electrode in this or another array of the strand.

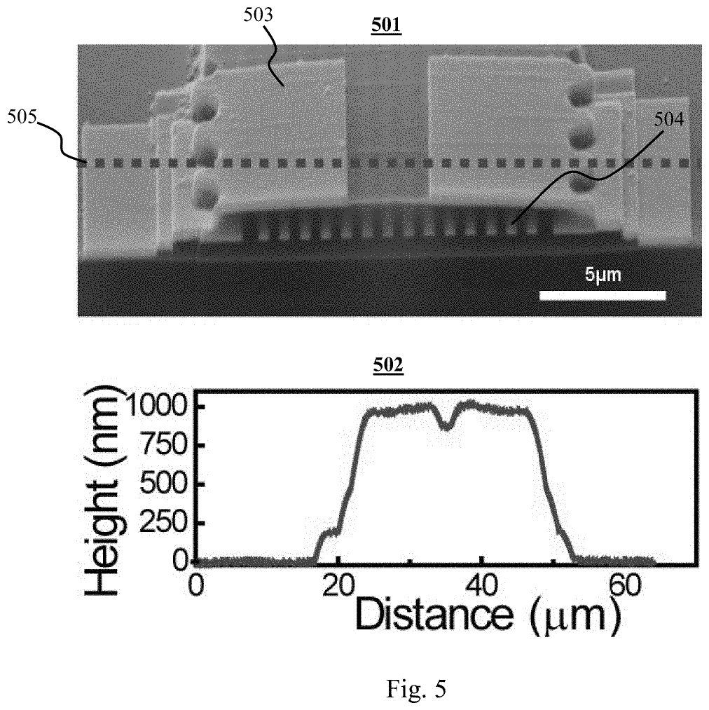

Referring now to FIG. 5, scanning electron microscopy (SEM) image 501 and graph 502 are shown. SEM 501 shows the relative height of electrodes 503 in relation to the width of the thread. Also shown are nanoscale wires 504 encased in the insulating dielectric. Graph 502 depicts a height profile of a thread, taken approximately along dotted line 505 using atomic force microscopy (AFM), showing the sub-micron thickness and fine layered structures of the cross-section.

In some embodiments, the electrodes are finished with gold film, but the electrodes may alternatively be fabricated with lower-impedance materials, including but not limited to platina (Pt), Iridium Oxide, (IrOx) or a conductive polymer such as poly(3,4-ethylenedioxythiophene) (PEDOT) or poly(3,4-ethylenedioxythiophene) polystyrene sulfonate (PEDOT:PSS). Electrodes should ideally be capable of detecting single unit action potentials with a high signal-to-noise ratio (SNR). In some embodiments, electrodes may include noise-suppression measures, for example a local reference electrode. In some embodiments, a system of the present invention comprises a computing device configured to perform offline analysis using a common mode reference to remove reference noise from the measured signal.

Exemplary electrode sizes are 5×8 μm and 12×15 μm, but electrodes may be larger or smaller as needed to measure the subject tissue. Typical electrodes could have a width (as measured horizontally in FIGS. 1-3) in the range of about 15 μm to 20 μm, or may be larger or smaller depending on the application. In certain instances, larger electrodes, for example electrodes having a surface area of about 500 μm² or about 1 mm², will lose the ability to resolve individual cells, but will be more capable of stimulation due to their increased capacity. In some embodiments, electrodes have a width in the range of about 500 nm to 1 μm. Electrodes can monitor an increased surface area by being longer (as measured vertically in FIGS. 1-3) and some electrodes may be in the range of about 20 μm-100 μm in length. In certain instances, larger electrode surface area will advantageously reduce impedance, but may introduce additional noise. In some embodiments, the impedance of each electrode at 1 kHz may vary between 100 kΩ-1.8 MΩ, but impedances in a range of about 50 kΩ to 2 MΩ are also envisioned.

Systems of the present invention may further include one or more rigid implantation assist devices, including for example a micro-shuttle device as shown in FIG. 9A. An SEM image of an exemplary micro-shuttle device is shown in view 902 of FIG. 9A, depicting shaft 924 and micro post 921. The micro-shuttle device is more rigid than the nanoelectronic threads of the present invention, and thus may be used to pierce the tissue and position the flexible nanoelectronic threads properly within the subject.

Further aspects of the present invention relate to a system and method for implanting intracortical probes comprising one or more nanoelectronic threads in the tissue of a subject. In certain instances, by themselves, ultra-flexible nanoelectronic threads are not rigid or strong enough to be inserted into brain tissue unassisted. In one embodiment, a micro-shuttle device is used, with a “needle-and-thread” strategy to deliver the nanoelectronic threads to the desired location and depth in tissue. Referring now to FIG. 9A, a micro shuttle 924 having micro post 921 engages with a hole 922 fabricated at the end of nanoelectronic thread 923. Examples of holes fabricated at the ends of nanoelectronic threads may be found in FIG. 9B. In some embodiments, micro shuttles with a diameter smaller than about 10 μm are used.

One alternative system and method of implantation is shown in FIG. 9C, which is divided into first and second steps 903 and 904. The system and method of FIG. 9C implements implantation with a high insertion speed in order to minimize surgical injuries. Because brain tissues are viscoelastic, a fast insertion speed can mitigate tissue dimpling and bleeding during the implantation. In one embodiment, the system comprises a multi-thread probe 931 having one or more nanoelectronic threads 938 and a rigid implantation assist device comprising a micromanipulator and a linear actuator. In one embodiment, the linear actuator comprises a solenoid 934 having a plunger 939 and an actuating member 935. In one embodiment, the micromanipulator comprises a linear guide 933 having a carrier block 940. In one embodiment, the linear guide comprises a mechanical stop 936. In the first step 903, a multi-thread probe 931 having one or more nanoelectronic threads 938 surrounded by or otherwise attached to a micro shuttle is mounted to a micromanipulator in parallel with a linear guide 933 having a carrier block 940. The micro shuttle may be attached to the nanoelectronic threads for example by a bio-dissolvable adhesive, including but not limited to PEG. The carrier block 940 is driven toward the tissue 937 by a linear actuator, for example a solenoid 934 having a plunger 939 and an actuating member 935. When the solenoid 934 is activated, the actuating member 935 pushes the carrier block 940 along the linear guide 933, driving the nanoelectronic threads 938 and the micro shuttle into the tissue 937. The carrier block 940 may be stopped at a predetermined point along the linear actuator by a variety of means, including but not limited to a mechanical stop 936. In some embodiments, mechanical stop 936 comprises a rubber O-ring. The stop has the effect of halting the implantation of the nanoelectronic threads into the tissue at a desired depth.

In some embodiments, the system of the present invention further comprises one or more controllers, connected to supply power and signals to, and to measure signals received from, probes of the present invention. In one embodiment, a controller is connected to a wired communication port of a probe, but in another embodiment the connection may be implemented via a wireless link. Power may be supplied to the controller via wires or wirelessly, and the controller may be positioned in vivo or ex vivo.

Systems of the present invention may further comprise one or more signal processing modules including but not limited to filtering, amplification, storage, and analysis modules, connected via wires or wirelessly to one or more probes. In some embodiments, the various signal processing modules are implemented as dedicated hardware circuitry, but the signal processing functions may also be implemented as software on a computing device. The purpose of the signal processing modules is to generate data and draw inferences from the measurements gathered from the various probes of the present invention. Filtering modules may include, but are not limited to high-pass, low-pass, or band-pass filters, Kalman filters, or any other filtering module used in the art. Amplification modules of the present invention may comprise one or more operational amplifiers or transistors, or may alternatively accomplish amplification through software means such as multiplication of analog values to add gain to some or all of the signals received. Storage modules may include any suitable means of data storage, including but not limited to hard disk drives, solid state storage, or flash memory modules.

In some embodiments, one or more of the threads of the present invention may be used for electrochemical sensing applications. For example, one or more electrodes on a thread may be coated with a capture agent capable of binding with a biochemical species of interest. Binding of the biochemical species to the capture agent results in a measurable change in conductance in the electrode. In this way, signal measurements from the electrodes on a nanoelectronic thread may be used to detect the presence, absence, or concentration of a chemical compound.

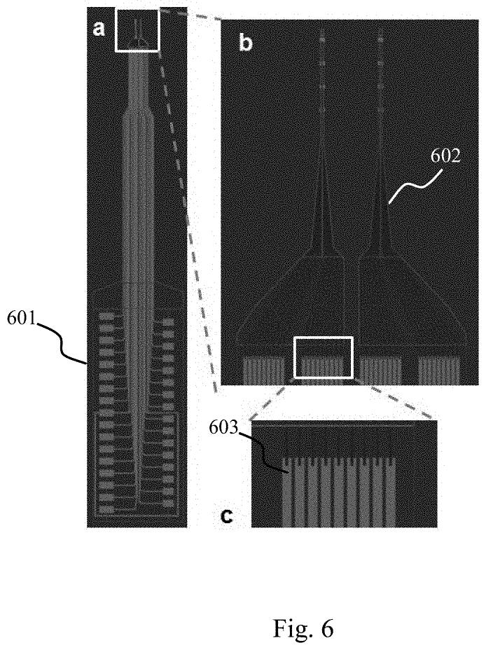

The present invention further includes a method of making an intracortical probe comprising one or more nanoelectronic threads. In one embodiment, a hybrid method involving both EBL and optical lithography is used to fabricate nanoelectronic thread devices with high throughput. EBL is used to define the implanted section (the “thread”) where dimensional constraints are more stringent, and photolithography with a more relaxed resolution requirement is used for larger structures that are not intended to be implanted in tissue. An example is shown in FIG. 6, wherein the nanoscale wires 602 (blue) are fabricated with EBL and the wider traces 601 (green) are fabricated with conventional optical lithography. The two methods are overlaid at the interconnects 603 between the finer nanoscale wires 602 and the thicker external traces 601. In one embodiment, EBL and photolithography are intentionally overlapped by at least 4 μm in all directions in all layers.

Different components may be constructed with different EBL techniques, wherein each production step is individually optimized for high yield, throughput, and fabrication resolution. In one embodiment, the nanoscale wires and electrodes are produced using standard EBL with poly(methyl methacrylate) (PMMA) as the positive resist followed by metallization and lift-off. Nanoscale wire width and pitch are typically minimized, but may be optimized to accommodate for stitching errors, and in one embodiment nanoscale wires are fabricated with a minimum line width of 200 nm and a pitch of 400 nm, in order to accommodate stitching errors of 40-50 nm and to improve fabrication yield.

One or more insulation layers may be fabricated using non-standard EBL, using a negative resist, including but not limited to PMMA or SU-8. SU-8 requires a very low exposure dose of less than 4 microcoulombs per square centimeter (μC/cm²) and therefore a controlled dose of EBL is necessary to achieve the desired resolution and thickness. In one embodiment, EBL with 0.5 μC/cm² was used. In another embodiment, EBL with 2.0 μC/cm² was used.

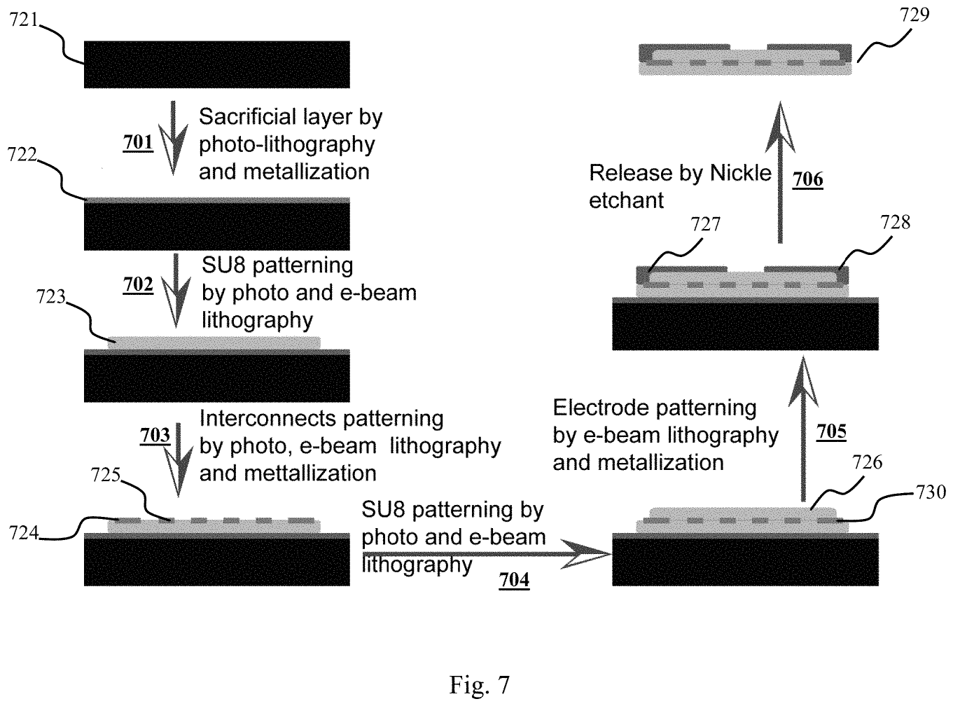

Referring now to FIG. 7, a diagram of an exemplary fabrication process is shown. In a first step 701, a sacrificial layer 722 is deposited on a silicon substrate 721. The sacrificial layer may be made of any appropriate material, including but not limited to nickel. Next, in step 702, insulating layer 723 is patterned on top of sacrificial layer 722 using photo lithography and EBL. In some embodiments, insulating layers of the present invention comprise SU-8, but other appropriate materials may be used. In some embodiments, all insulating layers have similar composition to one another, but in other embodiments, individual insulating layers may differ in their materials. Next, in step 703, nanoscale wires 724 and 725 are patterned on top of first insulating layer 723. In step 704, second insulating layer 726 is patterned on top of first insulating layer 723 and nanoscale wires 724 and 725. In the embodiment of FIG. 7, nanoscale wires 724 and 730 are exposed, while the remaining nanoscale wires are surrounded by first and second insulating layers 723 and 726. Next, in step 705, electrodes 727 and 728 are patterned on top of second insulating layer 726. Electrodes may be patterned by a variety of manufacturing processes, including but not limited to EBL and metallization. As shown, electrodes 727 and 728 are in electrical contact with nanoscale wires 724 and 730, respectively. Finally, in step 706, finished nanoelectronic thread 729 is released from silicon substrate 721 when sacrificial layer 722 is removed, for example with an etchant.

When manufactured with sub-micron thickness, certain embodiments of the invention exhibit ultra-flexibility that precludes free standing in air. FIG. 8A and FIG. 8B depict the flexibility of the individual nanoelectronic threads. System view 801 shows the region occupied by detail view 802, and connector 803. Detail view 802 shows that the individual threads 804 are flexible. Various embodiments may be further understood with reference to FIG. 8B, which depicts closer detail views of linear (810), oversampling (811), and tetrode (812) arrays floating in water after release from their respective substrates used in manufacturing. 810, 811, and 812 correspond roughly to FIG. 1, FIG. 2, and FIG. 3, respectively.

Another aspect of the present invention is a method of implanting intracortical probes comprising one or more nanoelectronic threads in the tissue of a subject. A method of implantation using a device such as the one depicted in FIG. 9A may begin by attaching a flexible probe to a rigid implantation assist device at an attachment point, for example inserting the post 921 at the end of a shuttle 924 into a hole 922 at the tip of a nanoelectronic thread 923. The shuttle 924 may then be inserted into the surrounding tissue, conveying the nanoelectronic thread into position within the tissue. Once the nanoelectronics thread is in place, the shuttle 924 may be withdrawn, leaving the thread 923 in place. A SEM image of an exemplary micro shuttle is shown in 902 of FIG. 9A. Larger micro shuttles risk dragging the nanoelectronic thread probe out during extraction due to the higher capillary force. The use of micro shuttle devices and thread smaller than about 10 μm has a further benefit in that it reduces the surgical footprint to subcellular dimensions, which forms tight tissue integration immediately after implantation. Thus, in some embodiments, threads remain embedded in brain tissue even when gently pulled. The shuttle may be inserted into the tissue to implant the intracortical probe using any suitable method, including but not limited to manual insertion, or electronically or mechanically actuated insertion, for example using a solenoid or other linear actuator as shown in the device depicted in FIG. 9C.

In some embodiments, a solenoid may drive the one or more nanoelectronic threads into the tissue at a speed in a range of 1-10 m/s. The speed and position of the carrier block 940 may be monitored, for example using a digital encoder, a capacitive sensor, or other linear position monitoring means. Once the array of nanoelectronic threads 938 pierces the tissue 937, in some embodiments the micromanipulator may be employed to further change the implantation depth, at a much slower speed than the implantation speed of the linear actuator. In embodiments where a dissolvable adhesive is used, the method may include the step of waiting for the adhesive between the nanoelectronic threads and micro shuttles to dissolve, at which point the micro shuttles may be retracted from the tissue, leaving only the nanoelectronic threads behind, embedded in tissue 937 (as shown in 904). Although the linear actuator in the embodiment of FIG. 9C is a solenoid, it is understood that other linear actuators may be used, including but not limited to a voice coil actuator, other types of magnetic actuators, or pneumatic actuators.

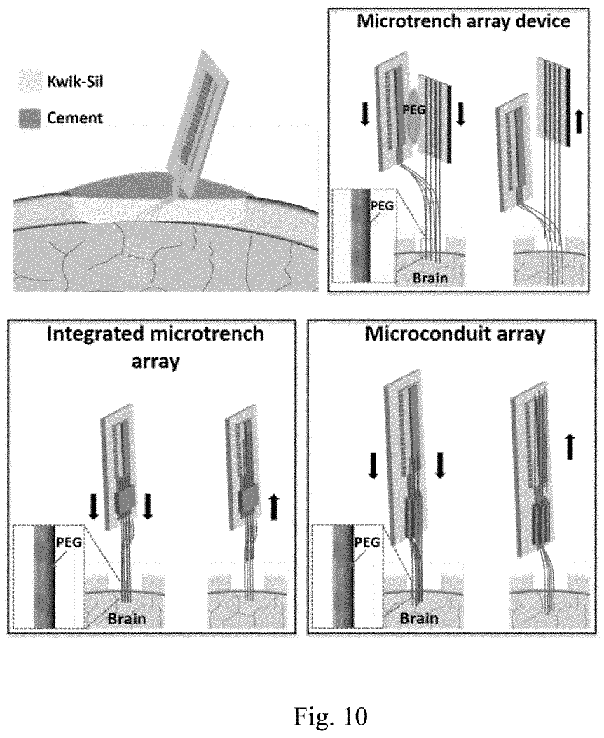

An alternative method of implantation involves parallel implantation of multiple nanoelectronic threads with the aid of an implantation assistance device as described above. Such methods allow for high throughput, parallel insertion of nanoelectronic threads with cellular-sized surgical footprints. As shown in FIG. 10, an exemplary multi-thread probe hosts 4-8 threads with similar recording capacity as a conventional microfabricated silicon probe but at drastically reduced thickness and rigidity. In the depicted example, each thread has a 4-layer layout, housing a linear array of 8-12 microelectrodes with a total thickness of 1 μm, width tapered from 65 μm to 30 μm, and implanted section of 1-2 mm in length. The inter-thread spacing may be uniform in one device and ranges from 150-400 μm depending on the design. One embodiment of the implantation scheme delivers all threads in parallel into the desired brain location and depth, while maintaining the mechanical and electrical integrity between the implanted section and the contact pad that is mounted on the skull after implantation. To achieve this goal, alternative implantation assist devices are shown in FIG. 10, including microtrench arrays and microconduit arrays. The nanoelectronic threads may be attached to the microwire array using a few-micron-thin layer of polyethylene glycol (PEG). The combined nanoelectronic threads and PEG have an extremely small profile, so surgical tissue displacements are typically dominated by the volumes of the shuttle devices, whose diameters are in the range of 25-50 μm. In some embodiments, the assembly is stereotaxically aligned and all threads are inserted simultaneously, as shown in the microtrench array device, integrated microtrench array, and microconduit array of FIG. 10.

In some embodiments, fine control over the implantation process is achieved by adjusting the molecular weight of the PEG coating, so as to precisely control the duration of adhesion between the shuttle-device and the nanoelectronic threads. In this way, the implantation method may target deep brain structures. The microtrenches and microconduits may be made from any suitable material, including but not limited to tungsten microwires or carbon nanotubes. Many of the delivery methods of the present invention use low-cost components that can be readily adapted to the implantation of other flexible neural implants.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of fault-tolerant and scalable parallel computing of the present invention. The following working examples therefore, specifically point out the preferred embodiments of the present invention, and are not to be construed as limiting in any way the remainder of the disclosure.

### Example 1: Nanoelectronic Thread-Based Electrode Array Fabrication

Different components of the probes of the present invention require different EBL techniques to construct, which were individually optimized for high yield, throughput and fabrication resolution. Standard EBL using PMMA as the positive resist followed by metallization and lift-off was performed to define the interconnects and the electrodes. The highest resolution was required for the nanoscale wire interconnect traces, which is determined by the EBL sample stage stitching error, because the length of the thread (about 1 mm) was much larger than the size of the writing field in the EBL tool used in this work (80 μm). A minimum linewidth of 200 nm (pitch of 400 nm) was used for the nanoscale wires to accommodate stitching errors at 40-50 nm and to improve fabrication yield. Non-standard EBL was performed to construct the insulation layers, for which both PMMA and SU-8 were evaluated as negative EBL resists. These two resists were chosen for their good insulation, excellent tensile strength after hard-baking, and tunable thickness depending on the exposure doses. Because PMMA requires a high dose of about 40,000 μC/cm² to crosslink, the required EBL exposure times are prohibitively long, even at high currents. Thus, SU-8 was chosen for higher fabrication throughput. However, SU-8 requires a very low exposure dose at less than 4 μC/cm², which makes the fabrication outcome highly sensitive to small changes in EBL conditions and to the design patterns. Therefore, the EBL dose was tested and controlled with 0.5 μC/cm² accuracy to achieve the necessary resolution and targeted thickness for every pattern.

A detailed step-by-step diagram is shown in FIG. 7. In the depicted example, fabrication of nanoelectronic threads was carried out on 100 mm silicon wafers (900 nm SiO₂, n-type 0.005 V·cm, University Wafer). First, EBL and metallization of 100 nm Au are used to define a set of global and chip alignment marks on substrate 721 for precise alignment among different fabrication steps. In step 701, photolithography and electron beam deposition were used to pattern a 100-nm nickel metal layer as sacrificial layer 722 that will be etched to release the flexible sections of the device at the end of the fabrication process. In step 702, a 300- to 500-nm layer of SU-8 photoresist 723 (2000.5; MicroChem Corp.) was deposited over the entire wafer and patterned by photolithography and EBL to define an insulating layer. Photolithography and metallization of 100 nm Au were then used to define the contact pads and interconnects outside the thread (not shown). In step 703, EBL and metallization was used to define the nanoscale wire interconnects 724 and 725 within the thread. Symmetrical Cr/Au/Cr or Cr/Pd/Cr (2-3/60-100/2-3 nm) metal was sequentially deposited to control the stress. In step 704, another 300- to 500-nm layer of SU-8 photoresist was deposited over the entire chip and patterned by photolithography and EBL to define the top insulating layer 726. In step 705, EBL and electron beam deposition of Cr/Au (2-3/100 nm) were used to define and deposit the electrodes 727, 728 that are individually addressed by contact pads through nanoscale wire interconnects. The wafer was then hard-baked at 180-190° C. for 5 hours to improve adhesion between two SU-8 layers 723 and 726. The wafer was then was cut to individual devices using a dicing saw (ADT, 7100 series). After fabrication, a 33-pin flexible flat connector (FFC) (series 502598, Molex) was mounted on the matching contact pads on the Si substrate. Finally, in step 706, the flexible section 729 was released from substrate 721 by etching off nickel layer 722 (Nickel Etchant TFB; Transene Company Inc.) for 2-4 hours at 25° C., rinsed and stored in distilled (DI) water before use.

The resulting fabrication yield was about 70%, with little variation among different design patterns that required fabrication resolution ranging between 200 nm to 400 nm. The fabrication process has been thoroughly examined, and yield loss is believed to be mainly due to fabrication defects. All fabrication procedures, including the four EBL steps, seven photolithography steps, and five metal deposition steps, were performed manually, which makes it difficult to completely avoid micro-particles and scratches especially on the long, narrow nanoscale wire interconnect traces. The EBL exposure time was relatively short. For example, an entire 4″ wafer that contains 8 nanoelectronic threads and a total of 256 electrodes typically requires exposure times of approximately 5 minutes for the insulating layers composed of SU-8, 2 hours for the nanoscale wire interconnects and 15 minutes for the electrode layers. However, the fabrication throughput was mostly limited by the two-step iterative alignment process during EBL that was necessary for sub-100 nm registration accuracy across the entire wafer. Despite the relatively low throughput compared to photolithography, the application of EBL greatly improved the fabrication resolution and reduced the probe's overall dimensions. It is believed that the fabrication yield, throughput and resolution can be further improved using more advanced EBL equipment.

The electrode impedance of the EBL defined probes was typically above 1 MΩ at 1 kHz, which was consistent with the small dimensions of the probes. Although single unit action potentials were detected with a sufficiently high SNR, the high impedance leads to a relatively high noise level and a narrow detection range. The signaling characteristics may be improved by using low-impedance materials for constructing the electrodes, such as Pt, IrOX and PEDOT coating. The noise levels during awake recording were elevated due to motion-induced coupling. This noise can be suppressed by using a local reference electrode or by offline analysis using a common mode reference.

Several technical challenges were overcome in the fabrication of probes, including precise inter-layer alignment across the entire wafer and the application of SU-8 as a sensitive negative e-beam resist to construct the insulation layers. A minimum linewidth of 200 nm was achieved (pitch of 400 nm) for the nanoscale wire interconnect traces and sub-100 nm inter-layer alignment. The total thickness of the nanoelectronic threads was determined mostly by the thickness of the two SU-8 layers, and was precisely controlled to be 0.8-1 μm by fine tuning the e-beam exposure dose of SU-8 with 0.5 μc/cm² accuracy.

As shown in FIG. 8C, the fabricated threads (shown at data point 821) have a significantly smaller footprint and cross section per electrode (cross-sectional area divided by number of electrodes) than comparable probes.

### Example 2: Shuttle Device Fabrication and Assembly

A straight segment of carbon fiber was attached to a stainless steel micro needle (prod #13561-10, Ted Pella, Inc.) for convenient handling. It was then cut to the designed length (2-3 mm) using a focused ion beam (FIB). An anchor post was micro-milled at the tip of the shuttle device using FIB to shape a well-defined micro-post (approx. 3 μm in diameter, 4 μm in height).

### Example 3: Shuttle Device Implantation

Wild type male mice (C57BJ/6, 8 weeks old, Taconic) were used in the experiments. Animals were housed at 22° C. (12 hour light/dark cycle, food and water ad libitum).

Mice were anesthetized using isoflurane (3% for induction and maintained at 1-2%) in medical grade oxygen. The skull was exposed and prepared by scalping the crown and removing the fascia, and then was scored with the tip of a scalpel blade. A 3 mm×3 mm square craniotomy was performed with a surgical drill over the somatosensory cortex. Dura mater was carefully removed to facilitate the delivery. After probe implantation, the remaining flexible segment of the probe, which connected the bonding pad on the substrate with the electrodes inside the brain, was routed to the edge of the cranial opening. The exposed brain was then protected by artificial cerebrospinal fluid (ACSF) and a coverslip #1 (Fisher Scientific) fit into the cranial opening. The space between the coverslip and the remaining skull was filled with Kwik-sil adhesive (World Precision Instruments). After the skull was cleaned and dried, a layer of low viscosity cyanoacrylate was applied over the skull. An initial layer of C&B-Metabond (Parkell Inc.) was applied over the cyanoacrylate and the Kwik-sil. A second layer of Metabond was used to cement the coverslip and the probe carrier chip to the skull.

In typical procedures, a flexible section of a nanoelectronic thread was placed on the brain surface where dura mater was removed. The shuttle device was mounted on a micromanipulator (MP-285, Shutter instrument) vertically, and positioned atop the engaging hole at the end of the probe. Referring to FIG. 9A, as shuttle device 924 traveled downward, anchor post 921 engaged hole 922 and pulled thread 923 into the brain tissue. Once the thread reached the desired depth, the shuttle device was retracted, and thread 923 was released and left embedded in the brain tissue.

Mice were allowed a 1-2 week recovery period after surgery. For recording, mice were head-constrained on a custom-made, air-supported spherical treadmill to allow walking and running, similarly to setups previously used for optical imaging. The treadmill was made of an 8-inch diameter Styrofoam ball (Floracraft) levitated by a thin cushion of air between the ball and a casting containing air jets. Voltage signals from the probes were amplified and digitized using a 32-channel RHD 2132 evaluation system (Intan Technologies) with a bare Ag wire inserted into the contralateral hemisphere of the brain as the grounding reference. The sampling rate was 20 kHz, and a 300 Hz high-pass and a 60 Hz notch filter were applied for single-unit recording. Mice and the amplifier were placed in a noise-attenuated, electrically shielded chamber. Impedance of the recording electrodes was measured using the same setup at 1 kHz prior to recording.

Two-photon (2P) imaging was performed in a laser scanning microscope (Praire Technology) equipped with a 20× water immersion objective (NA 1.0; Zeiss) and a Ti: sapphire excitation laser (MaiTai DS, Spectra-Physics) at a few weeks to months post-implantation. The laser was tuned to 810-910 nm for 2P excitation (power 3.0-50 mW, dwell time 4.0-6.0 μs). Fluorescence emissions were detected simultaneously by two standard photomultiplier tubes with a 595/50 nm filter (Semrock, US) for “red” fluorescence emission and a 525/70 nm filter (Semrock, US) for “green” fluorescence emission. Mice were anesthetized using isoflurane (3% for induction and 1.5% during experiment) in medical grade oxygen to maintain full immobility during imaging and placed in a frame that stabilized the head on the microscope stage. Anesthetized animals were given FITC-dextran (0.1 mL, 5% w-v, Sigma) retro-orbitally to label blood vessels prior to imaging. To facilitate imaging the probe-tissue interface beyond superficial cortical layers, the threads were doped with sulferhodamine 6G (Sigma) in the insulating layers and delivered at an angle of about 45 degrees with respect to the skull.

To prepare the histological samples, mice were given lethal intraperitoneal injections of 0.15 mL ketamine mixed with xylazine (10 mg/ml xylazine in 90 mg/mL ketamine) and then perfused intracardially with oxygenated, cold (˜4° C.) modified ACSF (2.5 mM KCl, 1.25 mM NaH₂PO₄, 25 mM NaHCO₃, 0.5 mM CaCl₂, 7 mM MgCl₂, 7 mM dextrose, 205.5 mM sucrose, 1.3 mM ascorbic acid, and 3.7 mM pyruvate) followed by 4% paraformaldehyde in 0.02 M phosphate-buffered saline (PBS). Brains were cryoprotected in a 30% sucrose/4% paraformaldehyde solution overnight. Tissue was sectioned into 20-50 μm slices perpendicular to the probe using a Leica CM1950 cryostat (Leica Microsystems). The slices were washed (3×5 min) and incubated in hot sodium citrate solution (85° C.-95° C., 0.01 M in H₂O) for 30 mins for antigen retrieval. Then, the slices were washed (3×5 min), incubated in blocking solution and permeabilized (0.5% Triton X and 10% normal goat serum (Sigma) in PBS) for 3 hours at room temperature, washed (4×5 min), and incubated in fluorophore conjugated antibodies for 24 hours at 4° C. Reagents used for neurons are (Millipore): Alexa Fluor 488 conjugated anti-NeuN antibody, clone A60.

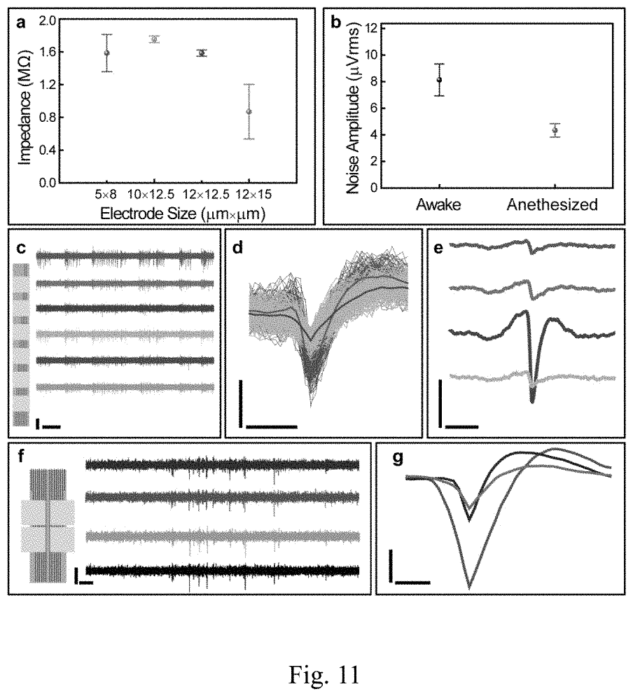

FIG. 11C and FIG. 11D show representative recording traces from multiple electrodes on a probe comprising multiple nanoelectronic threads and single-unit waveforms. FIG. 11C depicts representative unit activities recorded by an implanted nanoelectronic thread hosting a linear electrode array. The inset of FIG. 11C shows a schematic representation of the probe. FIG. 11D depicts superimposed spikes from the top electrode in FIG. 11C in the same recording session (5 minutes in total). Unit events are clustered into two distinct groups plotted in light gray and dark gray, with the mean waveforms for each plotted in blue and red, respectively. Adjacent electrodes along the thread at a separation of 80 μm were used to estimate the spike detection range, which depends on impedance and geometry. Detection range is a crucial parameter to determine the necessary electrode density for achieving complete detection of all enclosed neurons within a brain region. As shown in FIG. 11E, the same spikes were typically picked up by adjacent recording sites with strongly attenuated amplitudes, suggesting that the detection range is comparable to electrode separation. FIG. 11E is a 10 ms recording trace from FIG. 11C highlighting that a single spike was picked up by adjacent electrodes with attenuated amplitudes. Consistent with this detection range, the electrodes recorded temporally correlated spikes from the four “tetrode-like” electrodes in one group that were densely packed within an area of 18 μm×12 μm (FIG. 11F). The inset of FIG. 11F shows a schematic representation of the tetrode array.

The spatial-temporal correlation of the four electrodes improved the detection and sorting fidelity of single-unit action potentials (FIG. 11G) analogous to tetrodes. In addition, this scalable architecture enabled simultaneous detection at multiple brain depths. FIG. 11A shows in-vivo impedance at 1 kHz for electrodes with different dimensions. Twenty samples were taken for each dimension. FIG. 11B shows in-vivo measurement noise (RMS) of the smallest electrode (5 μm×8 μm) at anesthetized (right, 4.3 μV median) and awake (left, 8.1 μV median) measurements (bandwidth: 0.5 Hz to 7.5 kHz).

### Example 4: Microtrench Array Fabrication

Straight Tungsten microwires of a variety of diameters from 25-50 μm (W5607, Advent Research Materials) were manually cut to the desired length of 4-6 mm. Microtrenches of desired width and pitch were microfabricated on silicon substrates (4″ wafer, 900 nm SiO₂, n-type 0.005 V*cm, University Wafer) using standard planar photolithography. SU-8 photoresist (SU-8 2075, MicroChem Corp.) was spun on the silicon substrate at 4000 rpm for 45 seconds, followed by the standard SU-8 photolithography process which defined microtrench structures with a height of 60 μm. After hard baking (180° C., 3 hours), the 4″ wafer was cut to small rectangular pieces using a dicing saw (7100 Dicing system, ADT), which provided the carrier chip for shuttle devices. The width of the shuttle device carrier chip matched the width of the multi-shank thread substrate at 2-4 mm, and the length was about 3 mm, which provided sufficient margin for microwire alignment and convenience of handling. Tungsten microwires were placed into the trenches manually, resulting in a linear array of microwires at the pitch predefined on the carrier chip. The microwires protruded from the edge of the carrier chip by a few millimeters, where the ultra-flexible segment of nanoelectronic thread is attached. A thin layer of super glue (Loctite) was used to fix microwires in the trenches on the carrier chip without overflowing. Small drops of epoxy (Loctite) were placed on all four corners of the carrier chip to act as spacers between the chip and the probes when assembled together face-to-face. The shuttle device was then baked at 180° C. for 30 minutes for the epoxy to cure. Electrochemical etching (0.8 M KOH, graphite as anode, at 2.5 V for 30 seconds) was performed after the assembly procedure to sharpen the tungsten microwires.

Nanoelectronic threads were microfabricated using planar photolithography and multi-layer architecture, and diced to individual devices. A connector to external I/O was mounted on the contact pads and the flexible segment was released from the substrate by etching off the sacrificial layer. After the nanoelectronic thread was rinsed in phosphate-buffered saline (PBS) and allowed to dry in air, a shuttle device was placed on the nanoelectronic thread. The shuttle device was manually aligned under a stereomicroscope (A605, Leica microsystems), so that the lateral position of individual microwires matched the positions of nanoelectronic threads, and the microwires protruded from the end of the nanoelectronic threads by 10-20 μm. A customized clip was placed to gently press together the shuttle device carrier chip and the substrate, while a small gap between them was created by the epoxy drops at the corners of the shuttle device to protect the threads. After being rinsed in distilled water, the thread-shuttle device pair was dipped into a PEG solution (4% w/v, molecular weight of 4-5000 kDa, Alfa Aesar) and slowly pulled out so that the surface tension at the solution-air interface aligned the threads with the microwires. A 34-gauge syringe needle was used to manually assist attaching the threads onto the microwires at the air-solution interface. After the microwires and threads were aligned and pulled out of the solution, additional PEG was applied between the shuttle device carrier chip and the thread substrate to attach them together. The clip was then released and the assembled device was allowed to dry in air before implantation.

### Example 5: Intregrated Microtrench Array Fabrication

Methods of fabricating the integrated microtrench array (see FIG. 10) differ slightly from the methods described above. After the fabrication of the nanoelectronic threads, another photolithography process was applied to define microtrench arrays. SU-8 photoresist (SU-8 2075, MicroChem Corp.) was spun on the substrate at 4000 rpm for 45 seconds, followed by the standard SU-8 photolithography process which yielded a microtrench array with a height of 60 μm (same procedure as the fabrication of microtrenches on a separate silicon substrate). After hard baking (180° C., 3 hours), individual threads were cut and connectors to external I/O were mounted on the contact pad. Pre-cut straight tungsten microwires of a variety of diameters from 25-50 μm (W5607, Advent Research Materials) and length of 4-6 mm were manually placed into the microtrenches. The bottoms of the microwires were aligned to the end of the thread while the tops of the microwires were at least 2 mm lower than the bottom of the contact pad. The microtrenches were then partially filled with PEG solution to fix the microwires in place. An additional silicon chip was placed on top of the microtrenches and fixed on the nanoelectronic thread substrate by epoxy, which acted as a cap to restrain the microwires from moving out-of-plane during retraction. The bottom of the assembled chip was then soaked in Ni etchant (TFB, Transene Co. Inc.) for 2 to 4 hours at 25° C. to release the flexible segment of threads. After being rinsed in distilled water, the threads were attached to the microwires in PEG solution using the same procedure as described above. The assembled device was allowed to dry in air for implantation.

### Example 6: Fabrication of Microwire Arrays Guided by Microconduit Arrays

Methods of fabricating microwire arrays guided by microconduit arrays differ further from the methods described above. PTFE tubes (sub-lite-wall tubing, Zeus) of outer diameter 200 μm, inner diameter 100 μm and length of 6 mm were manually stacked to form a variety of structures, including linear, rectangular and triangular arrays. Epoxy (Loctite) was applied to permanently fix the structures. Pre-cut straight tungsten microwires as used in previous methods were inserted into a selection of the conduits. The microwires protruded from the tube edge by 3-5 mm on both ends while PEG solution was applied at one end of the conduits to temporarily fix the microwires. The microconduit array was then mounted on the nanoelectronic thread substrate using Epoxy (Loctite), and aligned under a stereomicroscope so that the lateral position of individual microwires best matched the positions of threads and the microwires protruded from the ends of the threads by 50-100 μm. The assembled device was then partially soaked in Ni etchant (TFB, Transene Co. Inc.) for 2 to 4 hours at 25° C. to release the flexible segment of threads. After being rinsed in distilled water, the threads were attached to the microwires in PEG solution as described above. For some structures, manual manipulation of the threads using a 34-gauge syringe needle was necessary to attach threads to the designated microwires. The assembled device was allowed to dry in air for implantation.

### Example 7: Linear Array Implantation with Assistance of Microwire Arrays

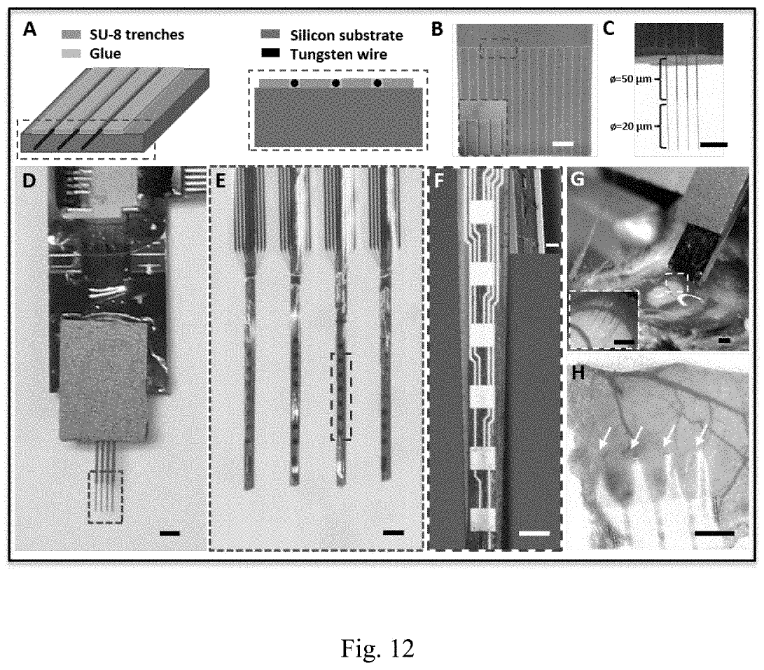

Parallel implantation of nanoelectronic thread linear arrays was demonstrated with the assistance of microwire arrays. In order to conveniently and precisely align microwires with threads, a microtrench array was fabricated on a shuttle device base chip (FIG. 12A) with a pitch matching that of the nanoelectronic threads to house microwires. The width of the microtrenches was designed to be 5 μm larger than the microwire diameter, which was 25-50 μm, so that the microwires could be placed into the trenches with little resistance, and at the same time be constrained to retain the pitch of the trenches (FIG. 12B). FIG. 12C and FIG. 12D show a representative shuttle device array for a 4-thread probe and an assembled nanoelectronic thread-shuttle device pair, in which the threads were attached onto microwires using dissolvable adhesive PEG. The microwires maintained straightness and the predefined pitch, and all electrodes on the threads had similar orientation after attaching (FIG. 12E). Individual threads formed nearly conformal attachment to the microwires without visible layers of PEG encapsulating the electrodes (FIG. 12F). Scanning electron microscopy (SEM) shows the submicron thickness of PEG between threads and microwires (FIG. 12F inset), which was favorable for minimizing the surgical footprint. The thread-shuttle device pair was inserted into the designated regions in a mouse brain using a regular stereotaxic setup, and the shuttle device was retracted after the PEG dissolved (FIG. 12G). Owing to the precise alignment of microwires prior to surgery, the implanted threads had nearly uniform pitch (FIG. 12H). Little bleeding and swelling were observed during and after insertion owing to the cellular-sized surgical footprints (FIG. 12H). The scale bars of FIG. 12 are: 500 μm (FIG. 12B, FIG. 12C, FIG. 12D, FIG. 12G, FIG. 12G inset and FIG. 12H); 100 μm (FIG. 12E); 50 μm (FIG. 12F) and 5 μm (FIG. 12F inset).

### Example 8: Implantation of Nanoelectronic Threads with Integrated Microtrenches

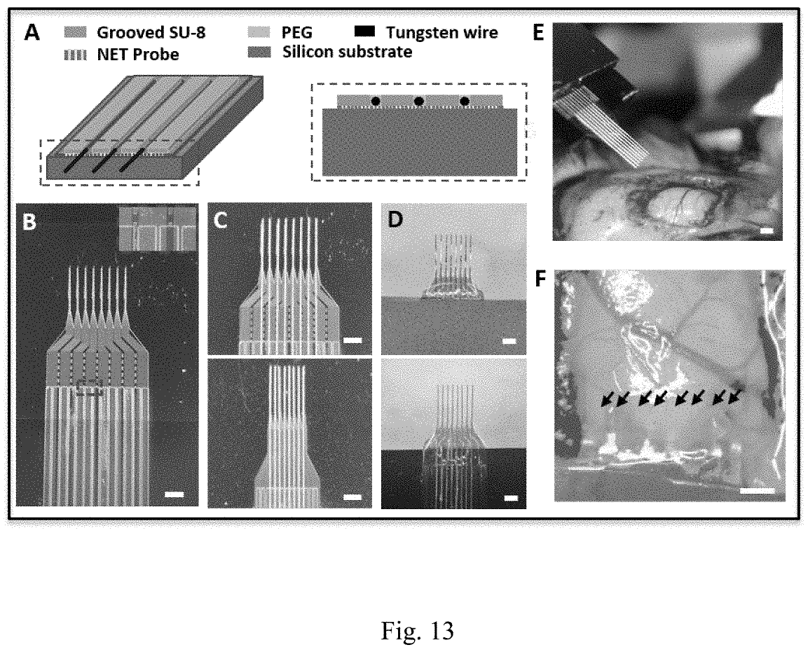

Parallel implantation of nanoelectronic thread arrays with integrated microtrenches was demonstrated, and is described with reference to FIG. 13. Microtrenches were fabricated directly on the thread substrate as shown in FIG. 13A. Implantation was accomplished without need for a separate shuttle device carrier chip or a second micromanipulator during the surgery. The microtrenches were designed to align with the positions of the thread shanks, a few mm above the implanted segments of the thread shanks so that the dimensions and mechanical rigidity of the implanted segment were not altered (FIG. 13B). The procedure to attach threads onto microwires using PEG solution is similar to the approach described in FIG. 12. Eight-thread probes with pitches of 250 μm and 150 μm were successfully attached to microwire linear arrays with matching pitches (FIG. 13C and FIG. 13D). Using simplified stereotaxic insertion and retraction procedures, 8-shank probes were implanted in the mouse cortex with designed spacing (FIG. 13E and FIG. 13F). Scale bars in FIG. 13 are 500 μm.

### Example 9: Implantation of Nanoelectronic Threads with Microconduit Arrays

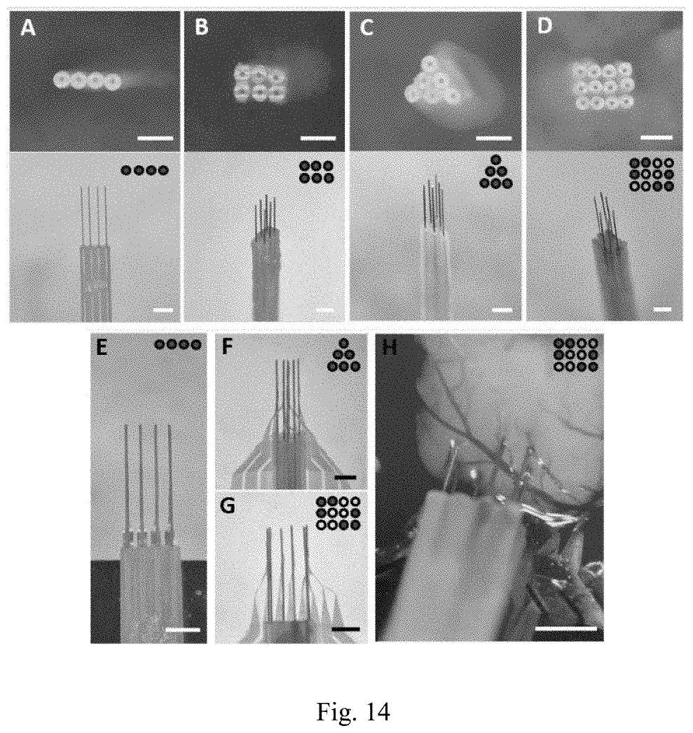

Parallel implantation of nanoelectronic thread arrays with microconduit arrays was demonstrated, and is described with reference to FIG. 14. While microtrench arrays typically require specialized equipment and expertise to fabricate, microconduit arrays made from off-the-shelf components were demonstrated facilitating the implantation of nanoelectronic thread based probes in a similar fashion. Micro-tubes with outer diameters close to the inter-thread spacing were selected and stacked to construct a variety of structures. Microwires were placed in all or a selection of the conduits to construct a microconduit array with customized patterns (FIG. 14A-FIG. 14D). Taking advantage of the ultra-flexibility of the threads, individual threads were manually routed to designated microwires (FIG. 14 E-FIG. 14G). In addition to linear arrays (FIG. 14E), conduits were arranged in triangular (FIG. 14F) and rectangular arrays, and threads were in all or a selection of conduits (FIG. 14G). Such versatile arrangements allowed the threads to have flexible spatial coverage for specific applications, such as simultaneous investigation of brain function that spans different brain regions. As an additional example, a customized pattern is shown in FIG. 14G for parallel delivery of 6 threads targeting two sites across a major surface blood vessel (FIG. 14H). Scale bars in FIG. 14 are 500 μm

### Example 10: Fine Control of Microwire Attachment Duration

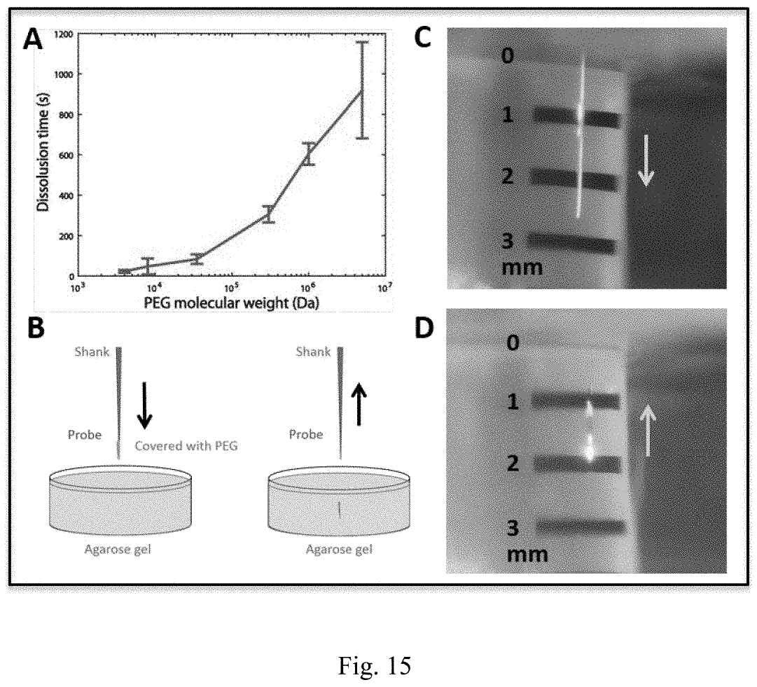

In the various parallel implantation solutions of the present invention, PEG solution was used to attach nanoelectronic threads to microwires, the PEG dissolving shortly after contact with brain tissue. The ability to control the release time of the threads is crucial for precisely controlling the insertion speed, which often affects the degree of surgical tissue damage. Typical insertion speeds for neural probes range from slow insertion at about 5 μm/s to fast insertion at about 1 mm/s, which requires attachment durations from 2 seconds to 400 seconds for targeting subcortical brain structures such as the hippocampus. The dissolution time of PEG in living tissue may be controlled by the molecular weight of PEG. For quantitative comparison, a constant concentration of PEG solution (4% w/v) was used, and the molecular weight was systematically varied to control the thread attachment duration. In-vitro tests were performed in PBS, and the dissolution time was found to be monotonically dependent on the molecular weight (FIG. 15A), with higher molecular weight leading to prolonged dissolution time. At molecular weight of 4 kDa, dissolution time was 10 seconds on average, while at 5000 kDa it increased to 900 s on average, which was sufficient for targeting subcortical structures a few millimeters below the brain surface even at slow insertion speeds. As further verification, an in vitro insertion test was conducted in agarose gel, while the insertion depth and speed were monitored in real time. A 1000 kDa PEG solution was used to attach a thread to a microwire, and the thread was inserted at 5 μm/s, to minimize acute tissue displacement. The thread remained attached on the microwire when the insertion depth reached 2 mm (FIG. 15C), comparable to the depth of the hippocampus in the rat brain. After waiting for 5 minutes and observing detachment of the nanoelectronic thread from the microwire, the microwire was retracted and the thread was left embedded in agarose gel. Consistently, using 1000 kDa PEG solution and the same insertion speed, implantation of nanoelectronic threads into the hippocampus of a mouse was demonstrated with an insertion speed of 10 μm/s.

### Example 11: Post-Mortem Evaluation of Tissue-Probe Interface

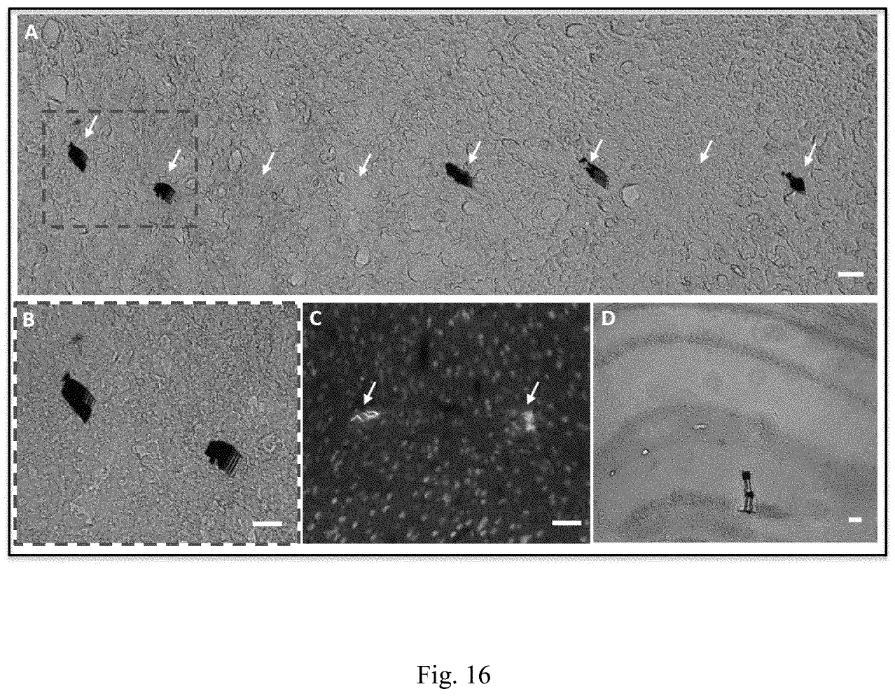

Two months after implantation, the tissue-thread interface was examined, with the results shown in FIG. 16. FIG. 16A-FIG. 16C show a representative tissue slice from a mouse cortex where an eight-thread nanoelectronic probe was implanted with inter-thread spacing of 250 μm. Neuronal density and tissue morphology appeared to be the same near and away from the threads, suggesting no neuronal degeneration was induced by the implantation. Brain slices in mice of which the hippocampus was targeted confirmed that diluted PEG at 1000 kDa provided sufficient adhesion to deliver threads to the desired depth (FIG. 16D). In addition, similarly to cortical implantation, no neuronal degeneration was observed for implantation into the hippocampus (FIG. 16D). The scale bars in FIG. 16 are 50 μm.

The above experiments demonstrated the possibility of integrating neural electrode arrays within a subcellular form factor and their implantation in a high-density, scalable manner. By applying nanofabrication techniques on unconventional substrate-less design of neural probes, the physical dimensions of neural probes were drastically reduced. Combining these nanofabricated ultra-flexible probes with minimally-invasive implantation methods at subcellular surgical footprints, a practical approach was developed to overcome current physical limits in the design and implantation of intracortical neural electrodes, which paves the way for full-coverage neural recording and complete circuit-level mapping of neural activity. More importantly, because little tissue response was observed for single or densely packed nanoelectronic threads after long-term implantation, the technique has the unique potential to chronically track the evolution of neuronal circuits for longer-term evaluation of brain development, for example learning and memory.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A probe, comprising:
a probe body having a distal end and a proximal end;
at least one flexible nanoelectronic thread extending from the distal end of the probe body, the nanoelectronic thread comprising:
at least one electrode positioned on a top surface of the nanoelectronic thread; and
at least one nanoscale wire having a first portion electrically connected to the at least one electrode and a second portion encapsulated by a dielectric insulator;
at least one external trace electrically connected to the at least one nanoscale wire at a proximal end of the at least one nanoelectronic thread; and
an interface connector near the proximal end of the probe body, electrically connected to the at least one electrode via the at least one nanoscale wire and the at least one external trace;
wherein the at least one nanoscale wire has a width less than 1 μm; and
wherein the nanoelectronic thread has a cross-sectional area that is less than 100 μm².

2. The probe of claim 1, wherein the at least one electrode comprises a plurality of electrodes positioned on the top surface of the flexible nanoelectronic thread, the plurality of electrodes being evenly spaced along a length of the nanoelectronic thread; and
wherein the at least one nanoscale wire comprises a plurality of nanoscale wires, the nanoscale wires having a pitch of less than 500 nm.

3. The probe of claim 2, wherein the plurality of electrodes are arranged in a linear array, and wherein the cross-sectional area of the flexible nanoelectronic thread is less than 10 μm².

4. The probe of claim 1, wherein the at least one electrode comprises at least one array of electrodes, the electrodes in the array being separated by a distance of less than 10 μm.

5. The probe of claim 1, wherein each of the electrodes has a sensing surface having an area less than 100 μm².

6. The probe of claim 1, wherein the at least one nanoelectronic thread comprises at least 8 nanoscale wires.

7. (canceled)

8. The probe of claim 1, wherein the at least one nanoelectronic thread further comprises a hole having a diameter of less than 10 μm.

9. The probe of claim 1, wherein the at least one external trace has a width greater than the width of the at least one nanoscale wire.

10. A method of fabricating a flexible probe, comprising the steps of:
patterning at least one external trace on a substrate using photolithography;
fabricating a flexible nanoelectronic thread using a process comprising the steps of:
depositing a sacrificial layer on a substrate;
depositing a first insulating layer on top of the sacrificial layer;
patterning at least one nanoscale wire on top of the insulating layer using electron beam lithography;
depositing a second insulating layer on top of the at least one nanoscale wire, thereby encapsulating at least a first portion of the at least one nanoscale wire and leaving at least a second portion of the at least one nanoscale wire exposed;
patterning at least one electrode on top of the second insulating layer, wherein the at least one electrode is in electrical contact with the second portion of the at least one nanoscale wire; and
removing the sacrificial layer and releasing the finished probe from the substrate;
wherein the electron beam lithography and photolithography are overlapped by at least 4 μm in all directions in all layers.

11. The method of claim 10, wherein the sacrificial layer comprises nickel and is removed with a nickel etchant, and wherein the first and second insulating layers comprise SU8.

12. The method of claim 10, wherein the first and second insulating layers comprise SU8.

13. The method of claim 10, wherein the electron beam lithography is controlled at a resolution of 0.5 μC/cm².

14. A method of implanting a flexible probe in a tissue of a subject, comprising the steps of:
attaching a flexible probe to a rigid implantation assist device at an attachment point;
inserting the flexible probe into a tissue of a subject by piercing the tissue with the rigid implantation device and inserting the rigid implantation assist device such that the attachment point is within the tissue;
detaching the flexible probe from the rigid implantation assist device; and
withdrawing the rigid implantation device from the tissue.

15. The method of claim 14, wherein the step of attaching further comprises the step of inserting a micro post on the rigid implantation device into a hole near a distal end of the flexible probe.

16. The method of claim 14, wherein the rigid implantation device and the flexible probe are inserted at a speed of 5 μm/second.

17. The method of claim 14, wherein the flexible probe is attached to the rigid implantation device via a chemical bond.

18. The method of claim 17, wherein the chemical bond comprises polyethylene glycol.

19. The method of claim 18, wherein the detaching step further comprises dissolving the polyethylene glycol.

20. The method of claim 14, wherein the rigid implantation assist device comprises a linear actuator and a carrier block slidably attached to a linear guide and fixedly attached to the flexible probe, and wherein the method further comprises actuating the linear actuator thereby implanting the flexible probe into the tissue.

21. The method of claim 20, wherein the linear actuator comprises a solenoid.

22. A probe made according to the method of claim 10.

23. A system for implanting a flexible probe in a tissue of a subject comprising:
the probe of claim 1, wherein the probe is flexible; and
a rigid implantation assist device, wherein the flexible probe is configured to engage the rigid implantation device.

24. The system of claim 23, wherein the rigid implantation assist device comprises a post configured to be inserted into a hole positioned at the distal end of the flexible probe.

25. The system of claim 23, wherein the rigid implantation device comprises a linear actuator and a carrier block slidably attached to a linear guide and fixedly attached to the flexible probe.
